(19) United States
(12) Patent Application Publication
(10) Pub. No.: US 2010/0028265 A1
(43) Pub. Date: Feb. 4, 2010

(54) WATER SOLUBLE MEDIA CONTAINING ANTI-MICROBIAL AGENTS

(76) Inventors: Steven A. Bolkan, Hopewell, NJ (US); Andrew Logan, JR., Warren, NJ (US); Melvin H. Czechowski, Landenberg, PA (US)

Correspondence Address: CHURCH & DWIGHT CO., INC., LAW DEPT. - PATENTS, 469 NORTH HARRISON STREET, PRINCETON, NJ, 08543-5297, US

(21) Appl. No.: 12/518,865

(22) PCT Filed: Dec. 13, 2007
(86) PCT No.: PCT/US2007/087465

§ 371 Date: Jul. 13, 2009

Related U.S. Application Data
(60) Provisional application No. 60/870,041, filed on Dec. 14, 2006.

(51) Int. Cl. A01N 55/00; A01N 25/02; A01N 25/06

(52) U.S. Cl. 424/43; 514/769; 514/63

(57) ABSTRACT

The present invention relates to method for killing various mold spores appearing on a surface by applying a composition comprising water soluble blast media and antimicrobial agent to the surface. The composition can be applied by a pressurized blasting apparatus or can be applied directly by any coating method. The composition can be immediately rinsed off the surface or applied directly to a surface and allowed to set and stay in contact with mold and mold spores.

### CROSS-REFERENCE TO RELATED APPLICATION

This application is related to U.S. Provisional Patent Application Ser. No. 60/870,041 filed Dec. 14, 2006 and takes priority therefrom.

### FIELD OF THE INVENTION

The present invention relates to methods of treating various mold spores appearing on a surface by applying a composition comprising alkalai metal bicarbonate and antimicrobial agent to the surface so as to kill and retard the growth of mold spores.

### DESCRIPTION OF THE PRIOR ART

Concern about indoor exposure to mold has been increasing as the public becomes aware that exposure to mold can cause a variety of health effects and symptoms. Molds can produce allergens that can trigger allergic reactions or even asthma attacks in people allergic to mold. Others are known to produce potent toxins and/or irritants. Potential health concerns are an important reason to prevent mold growth and to remediate/clean up any existing indoor mold growth.

Molds reproduce by making spores that usually cannot be seen without magnification. Mold spores waft through the indoor and outdoor air continually. When mold spores land on a damp indoor area, the spores may begin growing and digesting whatever media such as spores are growing on in order to survive. Molds gradually destroy the area on which the spores grow.

Mold spores can be found in the air and on nearly every surface in a home, but generally a consistent source of moisture is required for mold to grow. Molds can grow on virtually any organic substance, as long as moisture and oxygen are present. There are molds that can grow on wood, paper, carpet, foods, and insulation.

Many types of molds exist. Molds such as mold, fungus mold, and slime molds are most often found in areas that have high humidity levels such as bathrooms, kitchens, laundry rooms or damp basements (especially after flooding). Molds are a type of microscopic fungus that grow naturally indoors. The most common household mold types include Aspergillus, Cladosporium, Penicillium and Alternaria. Stachybotrys chartarum, often referred to as black mold, is less common than the molds listed above and is the type of mold commonly dealt with in home remediations.

In order to clean a solid surface so that such surface can again be coated such as, for example, to preserve metal against deterioration, remove graffiti from stone or simply to degrease or remove dirt from a solid surface, it has become common practice to use an abrasive blasting technique wherein abrasive particles are propelled by a high pressure fluid against the solid surface in order to dislodge previously applied coatings, scale, dirt, grease or other contaminants. Various abrasive blasting techniques have been utilized to remove coatings, grease and the like from solid surfaces. Thus, blasting techniques comprising dry blasting which involves directing the abrasive particles to a surface by means of pressurized air typically ranging from 30 to 150 psi, wet blasting in which the abrasive blast media is directed to the surface by a highly pressurized stream of water typically 3,000 psi and above, multi-step processes comprising dry or wet blasting and a mechanical technique such as sanding, chipping, etc. and a single step process in which both air and water are utilized either in combination at high pressures to propel the abrasive blast media to the surface as disclosed in U.S. Pat. No. 4,817,342, or in combination with relatively low pressure water used as a dust control agent or to control substrate damage have been used. Water for dust control has been mixed with the air either internally in the blast nozzle or at the targeted surface to be cleaned and such latter process, although primarily a dry blasting technique, is considered wet blasting inasmuch as media recovery and clean up is substantially different from that utilized in a purely dry blasting operation. The blast media or abrasive particles most widely used for blasting surfaces to remove adherent material therefrom is sand. Sand is a hard abrasive which is very useful in removing adherent materials such as paint, scale and other materials from metal surfaces such as steel. While sand is a most useful abrasive for each type of blasting technique, there are disadvantages in using sand as a blast media. For one, sand, i.e., crystalline silica, is friable and upon hitting a metal surface will break into minute particles which are small enough to enter the lungs. These minute silica particles pose a substantial health hazard. Additionally, much effort is needed to remove the sand from the surrounding area after completion of blasting. Still another disadvantage is the hardness of sand itself. Thus, sand cannot readily be used as an abrasive to remove coatings from relatively soft metals such as aluminum or any other soft substrate such as plastic, plastic composite structures, concrete or wood, as such relatively soft substrates can be excessively damaged by the abrasiveness of sand. Moreover, sand cannot be used around moving parts of machinery inasmuch as the sand particles can enter bearing surfaces and the like.

An alternative to sand as a blast media, particularly, for removing adherent coatings from relatively soft substrates such as softer metals as aluminum, composite surfaces, plastics, concrete and the like is sodium bicarbonate. While sodium bicarbonate is softer than sand, it is sufficiently hard to remove coatings from aluminum surfaces and as well remove other coatings including paint, dirt, and grease from non-metallic surfaces without harming the substrate surface. Sodium bicarbonate is not harmful to the environment and is most advantageously water soluble such that the particles which remain subsequent to blasting can be simply washed away without yielding environmental harm. Since sodium bicarbonate is water soluble and is benign to the environment, this particular blast media has also found increasing use in removing coatings and in cleaning dirt, grease and oil and the like from harder surfaces as well including steel and interior surfaces such as those which contact food such as in environments of food processing or handling.

Sodium bicarbonate is also a friable abrasive and, like sand, will form a considerable amount of dust during the blast cleaning process. To control the dust formed by the sodium bicarbonate blast media as it contacts the targeted surface, water is included in the pressurized fluid carrier medium. Thus, water can be used as the carrier fluid or, more preferably, injected into a pressurized air stream which carries the blast media from the blast nozzle to the targeted surface. Water as a means to control dust has been mixed with the air stream internally in the blast nozzle or into the air stream externally of the nozzle. The addition of water to the pressurized air stream has been very effective in controlling dust formed by the sodium bicarbonate blast media.

It has been suggested to use a blast cleaning process in which sodium bicarbonate is used as the blast media to clean food processing equipment as mentioned above. Commonly assigned U.S. Pat. No. 5,512,071 discloses such a process using sodium bicarbonate blast media and a surfactant which reduces the residues of the media that remain on the targeted surface. The present assignee also has been issued U.S. Pat. Nos. 5,423,146 and 5,939,357 directed to sprayable fertilizer compositions containing alkali metal bicarbonates and fungicidal agents for application to cultivated plants. The use of an alkali metal bicarbonate blast media for cleaning, killing, and/or preventing mold growth on building surfaces is not believed to have been suggested in the prior art.

### SUMMARY OF THE INVENTION

The present invention relates to method for treating various mold spores appearing on a surface by applying a composition comprising alkalai metal bicarbonate and one or more antimicrobial agents to the surface. The treatment will kill the mold or retard the growth of the mold spores and other microbes on the surface. The composition can be applied by a pressurized blasting apparatus or can be applied directly by coating. The composition can be applied to filter screens or applied directly to a surface and allowed to set and stay in contact with mold and mold spores. Also, the composition can be applied dry or wet to the surfaces.

### DETAILED DESCRIPTION OF THE INVENTION

The present invention relates to method for killing various mold spores appearing on a building surface by applying a composition comprising water soluble blast media and one or more antimicrobial agents such as fungicides and/or antibacterial agents to the contaminated surface. The composition can be applied by pressure, such as a pressurized blasting apparatus, a pressurized aerosol container, a spray bottle, and the like. The composition can be also applied directly by any coating method, such as spray coating, brushing, roller coating and the like. The composition can be also applied to filter screens or applied directly to any building surface and allowed to set and stay in contact with mold and mold spores. Also, the composition can be applied dry or wet to the surfaces.

The water soluble blast media typically will be in the form of a powder containing substantially singular abrasive particles have an average size range of from about 10 to 1,000 microns in diameter. Preferably, the blast media will comprise abrasive particles having an average size of from about 50-500 microns and wherein the amount of particles above 1,000 microns does not exceed about 1% of the total media. Water soluble blast media are advantageous since such blast media can be readily disposed of by a water stream. Water soluble blast media having a Mohs hardness of less than 5.0 are generally useful in this invention, in particular, for cleaning softer substrates. Non-limiting examples of water soluble blast media which can be utilized include the alkali metal and alkaline earth metal salts such as the chlorides, chlorates, carbonates, bicarbonates, sulfates, silicates, the hydrates of the above, etc. The preferred blast media are the alkali metal salts and, in particular, the sodium and potassium carbonates, bicarbonates and sulfates. The most preferred blast media are alkali metal bicarbonates as exemplified by sodium and potassium bicarbonates. Also useful in the present invention are sodium sesquicarbonate, natural sodium sesquicarbonate known as trona, sodium bicarbonate, sodium carbonate, potassium carbonate, potassium bicarbonate, sodium chloride and sodium sulfate which is described in U.S. Pat. No. 5,112,406. It is important to note that by water soluble is not meant completely water soluble as some salts and natural minerals such as trona may contain minor amounts of insoluble materials. For example, trona which is a natural sodium sesquicarbonate may contain up to 10 wt. % of insolubles. Thus, by water soluble is meant to include those materials which are substantially soluble in water and sufficiently soluble to leave a water soluble residue on a targeted surface. In general, the blast media will comprise over 85 wt. % of the antimicrobial composition. Preferably, levels of alkali metal carbonate or bicarbonate salts will comprise over 90 wt. % of the composition. Levels of alkali metal carbonates or bicarbonates ranging from 96 to 99 wt. % are exemplified.

To reduce residues of the blast media from remaining on the substrate surface, the blast media of the present invention can have surfactant incorporated therein. The surfactant which may be utilized can be anionic, nonionic, or amphoteric in nature. Mixtures of the various types of surfactant can be used. The surfactant may also aid in uniformly dispersing the antimicrobial agent within the blast stream if water is used at least in part in delivering the blast media to the surface to be treated.

Anionic surfactants appear to best reduce the residue formation of water soluble blast media components. Moreover, since most of the anionic surfactants are solids, such surfactants can be simply added as is to the blast media without adverse caking and agglomeration of blast media particles. Examples of suitable anionic surfactants are water-soluble salts of the higher alkyl sulfates, such as sodium lauryl sulfate or other suitable alkyl sulfates having 8 to 18 carbon atoms in the alkyl group, water-soluble salts of higher fatty acid monoglyceride monosulfates, such as the sodium salt of the monosulfated monoglyceride of hydrogenated coconut oil fatty acids, alkyl aryl sulfonates such as sodium dodecyl benzene sulfonate, higher alkyl sulfoacetates, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Examples of the last mentioned amides are N-lauroyl sarcosinate, and the sodium, potassium, and ethanolamine salts of N-lauroyl, N-myristoyl, or N-palmitoyl sarcosinate sold by W. R. Grace under the tradename “Hamposyl”. Also effective are polycarboxylated ethylene oxide condensates of fatty alcohols manufactured by Olin under the tradename of “Polytergent CS-1”.

Amphoteric surfactants are a well known class of surfactants which include the alkyl beta-iminodipropionates RN(C₂H₄COOM)₂ and the alkyl beta-aminopropionates RNHCH₄COOM where the alkyl group R contains 8 to 18 carbon atoms in both formulae and M is a salt-forming cation such as the sodium ion. Further examples are the long chain imidazole derivatives, for example, the di-sodium salt of lauroyl-cycloimidinium-1-ethoxy-ethionic acid-2-ethionic acid, and the substituted betaines such as alkyl dimethyl ammonio acetates where the alkyl group contains 12 to 18 carbon atoms. N-alkyl-2-pyrrolidones which are highly polar apiotic solvents, are also surface active and can be used. “Surfadone LP-100” from International Specialty Products has been found particularly useful.

Suitable nonionic surfactants include the polyoxyethylene-polyoxypropylene condensates, which are sold by BASF under the tradename “Pluronic”, polyoxyethylene condensates of alkyl phenols; polyoxyethylene condensates of aliphatic alcohols/ethylene oxide condensates having from 1 to 30 moles of ethylene oxide per mole of coconut alcohol; ethoxylated long chain alcohols sold by Shell Chemical Co. under the tradename “Neodol,” polyoxyethylene condensates of sorbitan fatty acids, alkanolamides, such as the monoalkoanolamides, dialkanolamides and the ethoxylated alkanolamides, for example coconut monoethanolamide, lauric isopropanolamide and lauric diethanolamide; and amine oxides for example dodecyldimethylamine oxide.

Fluorosurfactants can also be used. Preferably, the fluorinated surfactant for use in the present invention is a fluorinated hydrocarbon. The most preferred fluorinated surfactants for use in the present invention are Zonyl FSO Fluorosurfactant (described as a perfluoroalkyl ethoxylate) available from E. I. DuPont de Nemours & Co., Inc., and Fluorad FC-430 surfactant (described as a fluoroaliphatic polymeric ester) available from the Industrial Chemical Products Division of 3M.

The surfactant of the present invention can be incorporated into the water soluble blast media in a variety of ways. If solid, the surfactant can be mixed as is with the abrasive blast media particles. This is preferred and it has been found that the most useful surfactants for reducing residue formation are anionic surfactants which are mostly solid materials.

If the surfactant is liquid, the surfactant can be sprayed directly onto the blast media particles. While this method is the most direct way of incorporating the surfactant, the flow of the blast media through the metering means which meters the amount of abrasive particles into the fluid carrier stream may be adversely affected by incorporating the surfactant in this manner. Thus, the very fine particles of blast media may agglomerate and otherwise cake or bride together and render particle flow through a metering device difficult. Alternatively, the liquid surfactant can be sprayed onto the blast media particles, the coated blast media particles compacted and the compacted product which is formed regranulated into a surfactant-containing solid. Compacting may be performed by applying pressure to the surfactant-coated abrasive particles such as by continuously admitting the coated abrasive particles to a zone where the coated particles are subjected to pressure between two rolls running oppositely with respect to each other. A preferred means of compacting is by a roller compactor, wherein the particles are subjected to pressure between two rolls under an adjustable compacting pressure. An especially preferred compactor is the Fitzpatrick Co. “Chilsonater” roll compactor. The gap between the rolls, the amount of raw materials introduced to such a roll compactor and the compacting pressure can be adjusted to produce cohesive sheets or pellets of desired density and hardness. The sheets or pellets are then regranulated by any suitable granulating or crushing means. Preferably, the compacted sheets, pellets and the like are fed through a sieve crusher to force the compacted material through a sieve with meshes of a given size determining the particle size of the final product. Screening, if desired, can be performed by any suitable screening device.

Still further, the surfactant can be sprayed directly onto the abrasive blast media particles and the surfactant-coated particles then dusted with a very finely divided material to reduce the caking and bridging between the abrasive particles. Thus, finely divided fume silica, silicates such as clays, talc, mica, diatomaceous earth and metal silicates such as aluminosilicates including zeolites may be used for dusting the liquid surfactant-coated abrasive. Obviously, the addition of a significant amount of water insoluble additives reduces the advantages of the water solubility of the abrasive blast media with respect to disposal. Thus, the amount of dusting agent should be minimized. Inasmuch as the amount of surfactant to be included is minute, likewise the amount of the dusting agent required to maintain free-flow of the blast media should also be minimal.

Further, the surfactant can be added to any flow aids which are normally contained in blast media compositions by coating such materials prior to incorporation thereof with the abrasive particles.

An alternative to adding the surfactant to any of the solid materials which form the blast media is to add the surfactant to water if water is utilized as the primary fluid carrier medium. Thus, the surfactant can be added at the supply of water or can be added to the water stream at the blast nozzle. By incorporating the surfactant into the water stream, the disadvantages of adding additional water insoluble materials to the blast media is avoided and so is the agglomerating and caking, bridging and restriction to flow of the blast media avoided. Regardless of the method by which the surfactant is added to the blast media, it has been found that the amount of residues which remain on the target surface subsequent to blasting are drastically reduced upon the addition of the surfactant and any residues which do remain can be easily washed off with fresh water.

The amount of surfactant needed to provide reduced residue content and easily rinsed residues is extremely small in most cases and, thus, will range from about finite levels to about 3 wt. %, preferably about 0.05 to about 1 wt. %, and, more preferably, from about 0.05 to 0.5 wt. % of the abrasive blast media particles. It has further been found that the addition of the surfactant can actually aid in removing any dirt, grease or oil from the substrate. Nonionic surfactants appear to best provide the additional detersive action. Thus, it may be possible to provide several kinds of surfactants with the blast media including those most readily able to reduce residue formation such as anionic surfactants and those capable of enhancing the removal of dirt, grease or oil from the substrate. The surfactant advantageously solubilizes the dirt and grease allowing easier clean up and reduces the deflection of dirt from one surface to another.

The composition can also comprise flow aids. Such flow aids reduce caking of the water soluble blast media. Most preferably, the flow aid is a hydrophilic or hydrophobic silica, hydrophobic polysiloxane or mixture of such materials. These flow aids are typically added in amounts of 0.05 to 20%, preferably about 0.1 to 0.5% by weight relative to the total of abrasive particles. In fact, it has been found that the residues from the water soluble media which are formed are somewhat increased when the blast media composition contains a flow aid. Hydrophobic silica, unlike known hydrophilic silicas, is substantially free of non-hydrogen bonded silanol group and absorbed water. One preferred hydrophobic silica which may be utilized in the blasting media hereof is Aerosil R 972, a product which is available from DeGussa AG. This material is a pure coagulated silicon dioxide aerosol, in which about 75% of the silanol groups on the surface thereof are chemically reacted with dimethyldichlorosilane, the resulting product having about 0.7 mmol of chemically combined methyl groups per 100 m² of surface area and containing about 1% carbon. Its particles vary in diameter from about 10 to 40 nanometers and have a specific surface area of about 110 m²/gram. It may be prepared by flame hydrolysis of a hydrophilic silica as more fully described in Angew. Chem., 72, 744 (1960); F-pS 1,368,765; and DT-AS 1,163,784. Further details respecting such material are contained in the technical bulletin entitled “Basic Characteristics and Applications of AEROSIL”, DeGussa A. G., August 1986. The hydrophobic silica particles are admixed with the abrasive blasting media in the proportion of at least about 0.1 and up to about 1.0% by weight thereof. Another hydrophobic silica is Quso, marketed by DeGussa A. G.

Hydrophobic polysiloxanes, preferably non-halogenated polysiloxanes, suitable for use in the blasting media hereof are commercially marketed by Dow Corning and General Electric.

In addition to flow aids, the composition can also comprise anti-caking ingredients. Anti-caking ingredients are selected from particulate inorganic and organic compounds which are chemically unreactive with the other ingredients when the composition is in the form of a dry blend formulation. A selected compound preferably has a particulate size distribution less than about 100 microns in diameter.

Suitable anti-caking ingredients include silicious compounds, magnesium compounds, C₁₀-C₂₂ fatty acid polyvalent metal salt compounds, and the like. Illustrative of anti-caking ingredients are attapulgite clay, kieselguhr, silica aerogel, silica xerogel, perlite, talc, vermiculite, sodium aluminosilicate, ammonium carbonate, zirconium oxychloride, starch, sodium or potassium phthalate, calcium silicate, calcium phosphate, calcium nitride, aluminum nitride, copper oxide, magnesium carbonate, magnesium silicate, magnesium nitride, magnesium phosphate, magnesium oxide, magnesium nitrate, magnesium sulfate, magnesium chloride, and the like.

Preferred anti-caking ingredients include magnesium silicate and magnesium oxide. The use of magnesium silicate or magnesium oxide as an anti-caking ingredient has particular advantage for purposes of the present invention. Magnesium silicate and oxide contribute excellent anti-caking and free-flowing properties to an invention dry blend formulation.

The anti-caking ingredient normally is utilized in the least quantity which will affect the desired degree of anti-caking and free-flowing properties. Typically the anti-caking ingredient is incorporated in a dry blend formulation in a quantity between about 0.1-2 weight percent, based on the composition weight.

The antimicrobial agent of the invention can be a single compound or mixture of compounds which are included in a quantity which will provide a concentration between about 0.01-10 weight percent of the composition. The composition can be a dry blend powder or an water soluble formulation, suitable for spraying or coating.

The antimicrobial ingredient can be selected from a wide variety of organic and inorganic compounds or mixtures which are known and used in agriculture and horticulture applications, such as those listed in Agricultural Chemicals, Book IV, Fungicides, 1989 Revision (W. T. Thomson, Thomson Publications, Fresno, Calif. 93791).

The antimicrobial agent can be a fungicide. The general categories of fungicides include anilides, dithiocarbamates, halogenated derivatives, heterocyclic nitrogen derivatives, metallic derivatives, and the like.

Illustrative of fungicidal compounds are carbendazim, benomyl, thiophanate-methyl, thiabendazole, fuberidazole, dichlofluanid, cymoxanil, oxadixyl, metalaxyl, furalaxyl, benalaxyl, fenarimol, iprodione, procymidone, vinclozolin, penconazole, myclobutanil, pyrazophos, ethirimol, ditalimfos, tridermorph, triforine, nuarimol, triazbutyl, guazatine, propiconazole, prochloraz, flutriafol, chlortriafol, triadimefon, triadimenol, dichlobutrazol, fenpropimorph, fenpropidin, chlorozolinate, fenfuram, carboxin, oxycarboxin, methfuroxam, dodemorph, blasticidin S, kasugamycin, edifenphos, kitazin P, cycloheximide, phthalide, probenazole, isoprothiolane, tricyclazole, pyroquilan, chlorbenzthiazone, neoasozin, polyoxin D, validamycin A, repronil, flutolanil, pencycuron, diclomezine, phenazin oxide, nickel dimethyldithiocarbamate, techlofthalam, bupirimate, etaconazole, cypofuram, biloxazol, dimethirimol, fenapanil, pyroxyfur, polyram, maneb, mancozeb, captafol, chlorothalonil, anilazine, thiram, captan, folpet, zineb, propineb, binapactryl, nitrothalisopropyl, dodine, dithianon, fentin hydroxide, fentin acetate, tecnazene, quintozene, dichloran, copper oxychloride, copper sulphate, Bordeaux mixture, and the like.

Antibacterial compounds such as quaternary ammonium compounds are particularly useful in the composition of this invention. Examples of quaternary ammonium compounds include, but are not limited to, N-alkyldimethyl benzyl ammonium saccharinate, 1,3,5-triazine-1,3,5(2H,4H,6H)-triethanol; 1-decanaminium, N-decyl-N,N-dimethyl-, chloride (or) didecyl dimethyl ammonium chloride; 2-(2-(p-(diisobutyl)cresosxy)ethoxy)ethyl dimethyl benzyl ammonium chloride; 2-(2-(p-(diisobutyl)phenoxy)ethoxy)ethyl dimethyl benzyl ammonium chloride; alkyl 1 or 3 benzyl-1-(2-hydroxethyl)-2-imidazolinium chloride; alkyl bis(2-hydroxyethyl)benzyl ammonium chloride; alkyl dimethyl benzyl ammonium chloride; alkyl dimethyl 3,4-dichlorobenzyl ammonium chloride (100% C₁₂); alkyl dimethyl 3,4-dichlorobenzyl ammonium chloride (50% C₁₄, 40% C₁₂, 10% C₁₆); alkyl dimethyl 3,4-dichlorobenzyl ammonium chloride (55% C₁₄, 23% C₁₂, 20% C₁₆); alkyl dimethyl benzyl ammonium chloride; alkyl dimethyl benzyl ammonium chloride (100% C₁₄); alkyl dimethyl benzyl ammonium chloride (100% C₁₆); alkyl dimethyl benzyl ammonium chloride (41% C₁₄, 28% C₁₂); alkyl dimethyl benzyl ammonium chloride (47% C₁₂, 18% C₁₄); alkyl dimethyl benzyl ammonium chloride (55% C₁₆, 20% C₁₄); alkyl dimethyl benzyl ammonium chloride (58% C₁₄, 28% C₁₆); alkyl dimethyl benzyl ammonium chloride (60% C₁₄, 25% C₁₂); alkyl dimethyl benzyl ammonium chloride (61% C₁₁, 23% C₁₄); alkyl dimethyl benzyl ammonium chloride (61% C₁₂, 23% C₁₄); alkyl dimethyl benzyl ammonium chloride (65% C₁₂, 25% C₁₄); alkyl dimethyl benzyl ammonium chloride (67% C₁₂, 24% C₁₄); alkyl dimethyl benzyl ammonium chloride (67% C₁₂, 25% C₁₄); alkyl dimethyl benzyl ammonium chloride (90% C₁₄, 5% C₁₂); alkyl dimethyl benzyl ammonium chloride (93% C₁₄, 4% C₁₂); alkyl dimethyl benzyl ammonium chloride (95% C₁₆, 5% C₁₈); alkyl dimethyl benzyl ammonium chloride (and) didecyl dimethyl ammonium chloride; alkyl dimethyl benzyl ammonium chloride (as in fatty acids); alkyl dimethyl benzyl ammonium chloride (C₁₂-C₁₆); alkyl dimethyl benzyl ammonium chloride (C₁₂-C₁₈); alkyl dimethyl benzyl and dialkyl dimethyl ammonium chloride; alkyl dimethyl dimethylbenzyl ammonium chloride; alkyl dimethyl ethyl ammonium bromide (90% C₁₄, 5% C₁₆, 5% C₁₂); alkyl dimethyl ethyl ammonium bromide (mixed alkyl and alkenyl groups as in the fatty acids of soybean oil); alkyl dimethyl ethylbenzyl ammonium chloride; alkyl dimethyl ethylbenzyl ammonium chloride (60% C₁₄); alkyl dimethyl isopropylbenzyl ammonium chloride (50% C₁₂, 30% C₁₄, 17% C₁₆, 3% C₁₈); alkyl trimethyl ammonium chloride (58% C₁₈, 40% C₁₆, 1% C₁₄, 1% C₁₂); alkyl trimethyl ammonium chloride (90% C₁₈, 10% C₁₆); alkyldimethyl(ethylbenzyl) ammonium chloride (C₁₂₋₁₈); di-(C₈₋₁₀)-alkyl dimethyl ammonium chlorides; dialkyl dimethyl ammonium chloride; dialkyl dimethyl ammonium chloride; dialkyl dimethyl ammonium chloride; dialkyl methyl benzyl ammonium chloride; didecyl dimethyl ammonium chloride; diisodecyl dimethyl ammonium chloride; dioctyl dimethyl ammonium chloride; dodecyl bis(2-hydroxyethyl)octyl hydrogen ammonium chloride; dodecyl dimethyl benzyl ammonium chloride; dodecylcarbamoyl methyl dimethyl benzyl ammonium chloride; heptadecyl hydroxyethylimidazolinium chloride; hexahydro-1,3,5-thris(2-hydroxyethyl)-s-triazine; myristalkonium chloride; N,N-dimethyl-2-hydroxypropylammonium chloride polymer; n-alkyl dimethyl benzyl ammonium chloride; n-alkyl dimethyl ethylbenzyl ammonium chloride; n-tetradecyl dimethyl benzyl ammonium chloride monohydrate; octyl decyl dimethyl ammonium chloride; octyl dodecyl dimethyl ammonium chloride; octylphenoxyethoxyethyl dimethyl benzyl ammonium chloride; oxydiethylenebis(alkyl dimethyl ammonium chloride); quaternary ammonium compounds, dicoco alkyldimethyl, chloride; trimethoxysily propyl dimethyl octadecyl ammonium chloride; trimethoxysilyl quats, trimethyl dodecylbenzyl ammonium chloride; n-dodecyl dimethyl ethylbenzyl ammonium chloride; n-hexadecyl dimethyl benzyl ammonium chloride; n-tetradecyl dimethyl benzyl ammonium chloride; n-tetradecyl dimethyl ethylbenzyl ammonium chloride; and n-octadecyl dimethyl benzyl ammonium chloride.

Any other known or commercial antimicrobial agent can be added to the composition of this invention. A particularly useful agent is 2,4,4′-trichloro-2′-hydroxydiphenyl ether, which is also known as tricolosan.

The water soluble composition of the present invention comprising the water soluble abrasive particles, surfactant as described above and antimicrobial agent is useful for killing mold on surfaces. The composition can be applied to the desired surface by pressurized water or compressed air streams which contain water either added at the blast nozzle or externally therefrom so as to control dust formation. Blasting equipment for the bicarbonate blast media of the present invention is commercially available, for example, from International Surface Preparation Group, Inc. the Blast Media of Flow Rates Through the Blast Nozzle Typically Range from about 0.5 to 15, desirably from about 1.0 to 10.0 lbs per minute and under air pressures from 10 to 100 psi and water pressures for dust control typically ranging from about 10 psi and above.

The composition can also be incorporated into a pressurized can or a spray container for household use. Such compositions would be applied to the surface as an aerosol or finely divided atomized spray. Further, the compositions can be applied in a liquid carrier and simply brushed or rolled onto the desired surface. Alternatively, the composition may also be applied manually via a packaged shaker-type container wherein the fine particles of the composition are cast onto vertical or horizontal surfaces. The compositions, once applied, can be immediately rinsed off with water or allowed to set and dry.

The composition of the present invention can be applied to surfaces in which mold is already present or can be applied so as to prevent the growth of mold spores. The invention is particularly useful in killing and preventing the further growth of mold on any and all exterior and interior building surfaces. Such surfaces can be formed of any material onto which mold may grow, in particular when such surfaces become wet and can remain damp for a significant period of time. Thus, the composition can be applied to metal, masonry, stucco, plaster, wood, or plastics. Interior surfaces such as formed from ceramic tile, which is spaced by a plaster-type grout, can also be effectively treated with the composition of this invention. The composition can be applied to filters such as used to remove particulates from an air stream for heating or cooling an interior space. It is believed that application to the filters would kill any mold spores captured within the filter medium, thus preventing the spread of mold through the interior environment.

### Example 1

Table 1 illustrates useful compositions of the present invention. This list is exemplary and other equivalents can be substituted therefor.

TABLE 1

| FORMULA | A | B | C | D | E | F | G |
|---|---|---|---|---|---|---|---|
| 1 | 99.7 | 0.25 | — | — | — | — | — |
| 2 | 99.4 | 0.25 | 0.10 | 0.25 | — | — | — |
| 3 | 99.3 | 0.25 | 0.10 | 0.25 | 0.10 | 0.10 | — |
| 4 | 99.1 | 0.25 | 0.10 | 0.25 | 0.10 | 0.10 | 0.10 |

A. Sodium Bicarbonate
B. Silicon Dioxide (Sylox-15) or Sipernat 22S
C. Hamposyl L-95
D. Magnesium Oxide (Mag Chem 30)
E. Detergent Range Alcohol Ethoxylate (DRAE)
F. Quaternary Ammonium Chloride
G. Triclosan
Potassium Bicarbonate 0.5-50% by weight

Potassium bicarbonate can be substituted for about 0.5 to 50 weight percent of the sodium bicarbonate.

### Example 2

1.0% AEM 5772 (Aegis Antimicrobial agent)
0.05% Zonyl FSH (fluorsurfactant)
0.1% Safol 23 E7 (ethoxylated alcohol)

### 98.85% Water

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method for treating mold present on a building surface comprising: applying a composition containing an alkalai metal bicarbonate and an antimicrobial agent onto said surface.

2. The method according to claim 1, wherein the alkalai metal bicarbonate is sodium bicarbonate or potassium bicarbonate.

3. The method according to claim 1, wherein the composition is applied to said surface by a pressurized water or air stream.

4. The method according to claim 3, wherein the composition comprises at least 85 wt. % of said alkali metal bicarbonate.

5. The method according to claim 4, wherein the alkalai metal bicarbonate comprises about 90 to 99 wt. % of bicarbonate salt of sodium, potassium or mixtures thereof.

6. The method according to claim 1, wherein said composition further includes a surfactant.

7. The method according to claim 6, wherein the composition further comprises a flow aid.

8. The method according to claim 1, wherein the antimicrobial agent is a quaternary ammonium compound.

9. The method according to claim 8, wherein said antimicrobial agent is a mixture of at least two antimicrobial agents.

10. The method according to claim 1, wherein the composition is applied to the surface in a pressurized air stream.

11. The method according to claim 1, wherein the composition is applied to the surface as an aerosol.

12. The method according to claim 1, wherein said building surface comprises an interior building surface.

13. The method according to claim 12, wherein said surface is a filter for removing particulates from an air stream.

14. A composition for treating mold present on a surface of a building, said composition comprising at least about 85 wt. % of alkali metal bicarbonate and at least one antimicrobial agent.

15. The composition of claim 14 containing at least 90 wt. % of said alkali metal bicarbonate.

16. The composition of claim 14 further including a surfactant.

17. The composition of claim 16 further including a flow aid.

18. The composition of claim 14 wherein said at least one antimicrobial agent is a quaternary ammonium compound.

19. A composition contained in a pressurized gas or liquid carrier, said composition containing at least 85 wt. % alkali metal bicarbonate and an antimicrobial agent.

20. The composition of claim 19, wherein the alkali metal bicarbonate is sodium bicarbonate or potassium bicarbonate.
